(19) United States
(12) Patent Application Publication
(10) Pub. No.: US 2022/0230479 A1
(43) Pub. Date: Jul. 21, 2022

(54) INFORMATION PROCESSING SYSTEM FOR VEHICLE AND A METHOD FOR PROCESSING INFORMATION FOR VEHICLE

(71) Applicant: Continental Automotive GmbH, Hannover (DE)

(72) Inventors: Kuhan PALANIAPPAN, Singapore (SG); Zi Hui LAU, Singapore (SG); Fidel Jr Acaylar GAINSAN, Singapore (SG); Ernie Ray Plaza DUMANGAS, Singapore (SG); Wai Lynn HTUN, Singapore (SG)

(21) Appl. No.: 17/605,768

(22) PCT Filed: Apr. 23, 2020
(86) PCT No.: PCT/EP2020/061260

§ 371 Date: Oct. 22, 2021

(30) Foreign Application Priority Data
Apr. 24, 2019 (GB) 1905686.0

(51) Int. Cl. G07C 5/00; H04W 4/90; H04W 4/44; H04W 4/80; H04W 4/02

(52) U.S. Cl. CPC G07C 5/008; H04W 4/90; H04W 4/023; H04W 4/80; H04W 4/44

(57) ABSTRACT

A system and method for processing information after a traffic accident is provided herein. A gateway receives at least one type of health data collected by a group of electronic devices or sensors, to determine a condition of vehicle collision victim(s). In response to a vehicle crash signal, the gateway transmits the health data to an external party and/or triggers autonomous function of the vehicle to ensure safety of the victim(s) and adequate medical attention may be given to the crash victim(s).

### CROSS REFERENCE TO RELATED APPLICATIONS

This application is the U.S. National Phase Application of PCT International Application No. PCT/EP2020/061260, filed Apr. 23, 2020, which claims priority to Great Britain Patent Application No. 1905686.0, filed Apr. 24, 2019, the contents of such applications being incorporated by reference herein.

### FIELD OF THE INVENTION

This disclosure relates to information processing systems for vehicle and methods for processing information for vehicle. In particular, the present invention concerns information processing systems for vehicle and methods for processing information for vehicle which can be advantageously used upon a traffic accident situation. Further, the present invention concerns computer-readable storage mediums for implementing the afore-mentioned systems and methods.

### BACKGROUND OF THE INVENTION

In the event of traumatic event, for example traffic accident or vehicle collision, paramedics are usually the frontline emergency response team providing immediate medical attention to the victims, before the victims are transported to a communal healthcare centre, such as a hospital which are equipped with more sophisticated instruments to provide medical attention for complex injuries.

At times during the transportation between accident scene to hospital, wounded victims may experience medical complication. Consequently, timely and adequate medical attention given to the victims is crucial.

### SUMMARY OF THE INVENTION

A purpose of this disclosure is to provide a solution to improve crisis management during traffic accidents, by addressing lack of health data or medical history of accident victims communicated to emergency response team and/or medical communal centre in the event of traffic accidents, to provide timely and adequate medical attention to traffic accident victims. Another purposes is for avoiding vehicle chain collision, by incorporating autonomous driving function in response to traffic accident.

In the first aspect, an information processing system for vehicle is provided herein. A first electronic control unit may be equipped within a vehicle. The first electronic control unit may be configured to receive and transmit an accident-related data in relation to the vehicle. At least one gateway may be configured to receive the accident-related data from the first electronic control unit, to receive at least one health data of an occupant in the vehicle, the at least one health data stored in or collected by at least one of mobile device of the occupant and sensing device embedded in the vehicle, and transmit a first signal to a telematic control unit upon the receipt of the accident-related data. The telematic control unit configured to receive the first signal from the at least one gateway and transmit an alert together with a set of data which comprises at least a part of the health data or an access right to an external server comprising at least a part of the health data to a predetermined external agent upon the receipt of the first signal.

The at least one gateway may be further configured to transmit the at least a part of the health data to the telematic control unit.

The gateway may be further configured to transmit a second signal to a second electronic control unit upon the receipt of the accident-related data, and may further comprising the second electronic control unit may be configured to receive the second signal from the gateway and activate one or more autonomous driving functions for emergency situation upon the receipt of the second signal.

The autonomous driving function may be selected from a self-driving function, a self-parking function, a hazard light indicator function or any combination thereof.

The at least one health data may be transmitted via wired communication protocol or wireless communication protocol by the at least one of mobile device of the occupant and sensing device embedded in the vehicle to the gateway.

The accident-related data may comprises a vehicle collision information.

The mobile device of the occupant may be selected from a mobile communication device, a wearable device, or any combination thereof.

The mobile device of the occupant may be configured for pairing with the gateway, prior to receiving at least one health data of the occupant in the vehicle.

The pairing between the mobile device of the occupant and the gateway may be conducted by scanning an identification code of the occupant at a terminal electrically connected to the system of the vehicle.

The pairing between the mobile device of the occupant and the gateway may be conducted by tapping a radio frequency identification (RFID) card containing an unique identifier of the occupant.

The pairing between the mobile device of the occupant and the gateway may be conducted by entering an identification of the occupant by way of the mobile device.

The pairing between the mobile device of the occupant and the gateway may be configured to enable wireless communication health data between the mobile device of the occupant and the gateway upon successfully pairing.

The gateway may be configured to wirelessly communicate with an external server using a long-range communication mode.

The external server may be a computer-readable storage medium configured to store the health data from the gateway.

The sensing device may comprises a vehicle interior sensor, in particular, amperometric biosensor, blood glucose biosensor, potentiometric biosensor, conduct metric biosensor, thermometric biosensor, optical biosensor, fibre optic lactate biosensor, piezoelectric biosensors, immune-biosensors or any combination thereof.

The at least one health data may comprises pulse rate, body temperature, respiration rate, blood pressure, blood glucose level, electroencephalogram (EEG), electromyography (EMG) or any combination thereof.

The set of data may further comprises a positional data of the vehicle.

The set of data may further comprises the identification of at least one occupant in the vehicle.

In a second aspect of this disclosure, a method of processing information for vehicle is provided herein. The method may include receiving, by a first electronic control unit of the vehicle, an accident-related data in relation to the vehicle; transmitting, by the first electronic control unit, the accident-related data to at least one gateway of the vehicle; receiving, by the at least one gateway, at least one health data of an occupant in the vehicle from at least one mobile device of the occupant and sensing device embedded in the vehicle; transmitting, by the at least one gateway, a first signal to a telematics control unit of the vehicle; and transmitting, by the telematics control unit, upon the receipt of the first signal, an alert and a set of data comprising at least a part of the health data collected by the at least one gateway, or an access right to an external server, to a predetermined external agent.

The at least a part of the health data may be transmitted to the telematics control unit by the at least one gateway.

The method may further comprise transmitting, by the at least one gateway, a second signal to a second electronic control unit of the vehicle upon the receipt of the accident-related data, for activating one or more autonomous driving functions for emergency situation upon receiving the second signal.

The method may further comprise collecting, by the at least one gateway, a positional data of the vehicle upon the receipt of the accident-related data, wherein the set of data further comprises the positional data of the vehicle.

The method may further comprise collecting, by the at least one gateway, an identification of at least one occupant in the vehicle, upon the receipt of the accident-related data, wherein the set of data further may comprises the identification of at least one occupant in the vehicle.

The at least one health data may be transmitted via wired communication protocol or wireless communication protocol by the at least one of mobile device of the occupant and sensing device to the gateway.

In a third aspect of this disclosure, a computer-readable storage medium is provided. The computer-readable storage medium may store instructions that, where executed by one or more processors, cause the processors to perform operations of the method as disclosed above.

Advantageously this disclosure solves the problem of crisis management during accidents, by addressing lack of health data or medical history of accident victims communicated to emergency response team and/or medical communal centre in the event of accidents, to provide timely and adequate medical attention to accident victims through:
(i) compiling health data of occupant(s) from various sources to define identification of victim, health condition of victim after crisis and/or make available health history of victims;
(ii) processing the compilation of health data and provide a set of health data or health report to a predetermined external party in the event of accident to ensure timely and adequate medical attention may be given to victims
(iii) providing a position the location of vehicle after traffic-accident, such that emergency response team can be deployed to traffic-accident scene in a timely manner.

Another advantage of this disclosure is improvement of crisis management, by incorporating autonomous driving function in response to traffic accident, to avoid vehicle chain collision.

### BRIEF DESCRIPTION OF DRAWINGS

Other aspects will become apparent from the following description of embodiments with reference to the accompanying drawings in which:

FIG. 1 illustrates a block diagram of a system 100 according to an exemplary embodiment as disclosed herein.

FIG. 2 illustrates a block diagram 200 of a bio sensor gateway according to an exemplary embodiment as disclosed herein.

FIG. 3 illustrates a flowchart 300 of a method according to an exemplary embodiment as disclosed herein.

FIG. 4 illustrates an infrastructure 400 of the system in an exemplary embodiment

### DETAILED DESCRIPTION OF EMBODIMENTS

Hereinafter, an explanation of a system and method for processing health information in response to an accident or traumatic event will be discussed in details.

For clarity, the term “vehicle” or “vehicular” shall denote vehicles in general such as passenger automobiles including sports utility vehicles (SUV), and includes hybrid vehicles, electric vehicles, combustion, plug-in hybrid electric vehicles, hydrogen-powered vehicles and other alternative fuel vehicles (e.g. fuels derived from resources other than petroleum), public transportation such as buses, trucks and trains.

The term “health data” shall denote epidemiology information related to health conditions, reproductive outcomes, causes of death and quality of life. Consequently, the term “health data” of an occupant in a vehicle” denotes epidemiology information relating to a driver or passenger(s) within an interior of a vehicle. The term “occupant” and its grammatical variants therefor shall denote driver, passenger, public transport operator and or public transport commuter.

The term “accident-related data” shall denote a vehicle collision signal, indicating a traffic accident, for example the vehicle has collided with another vehicle, pedestrian, or some other objects.

The term “positional data” shall denote information relating to or determined by a geographical location of the vehicle.

The term “device” and its grammatical variants thereof shall denote an electronic or mechanical item made or adapted for a particular purpose, for example a “sensing device” shall refer to an electronic item adapted for detection.

The expression “mobile device” and its grammatical variants thereof shall denote a handheld item that is both portable and compact, for example a mobile communication device. In this sense, a “wearable device” denotes an electronic or mechanical item that is suitable for wearing. In the context used herein, “wearable device” may serve to function as a front end device to a remote system, such as a mobile communication device configured to synchronize with the wearable device using wireless technology, where the mobile communication device many include palmtop computers such as personal digital assistant (PDA), or smartphones operating on different types of operation systems, but aspect of the present invention are not limited thereto.

The term “pair” or “pairing” and its grammatical variants thereof shall denote a process of linking at least two device so that these devices may wirelessly communicate via short-range communication mode using radio waves signals.

The term “signal” and its grammatical variants thereof shall denote an electromagnetic weave, such as a radio or microwave, that has the function of transmitting data packets carrying information between different points or nodes of a system or network. The term “wireless signal” and its grammatical variants thereof shall denote an electromagnetic wave, such as radio or microwaves, that has the function of transmitting data packets carrying information between different points of a system or network.

Accordingly, the term “wirelessly” shall denote transmitting of or exchange of data packets between different points of a network or systems of networks using electromagnetic or radio wave, without cables. The term “wireless communication” or “wirelessly communicate” shall therefore, denote communication or exchange of data packets between different points of a network or systems of networks using wireless signals. Conversely, “wired communication” refers to the same activity of exchange of data packets over wires, for example cables.

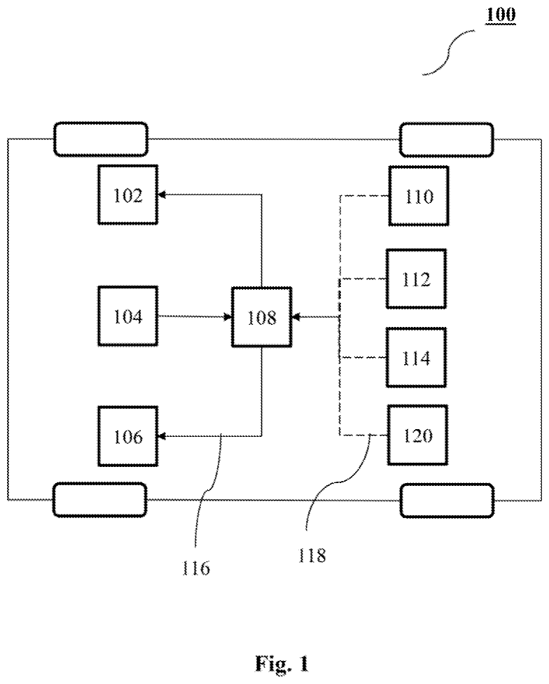

Referring to FIG. 1 which illustrates a system block diagram 100 in accordance to an exemplary embodiment as disclosed herein, at least one gateway 108 is electrically connected with a first electronic control unit 102, a second electronic control unit 104 and a telematic control unit 106 via a wired communication protocol 116. Suitable types of wired communication protocol 116 includes vehicle bus topologies, for example Controller Area Network (CAN) bus, Local Interconnect Network (LIN) bus, Flexray, Inter-integrated circuit (I2C), System Packet Interface (SPI) network.

The first electronic control unit 102 may be a vehicle crash management system within a vehicle. An example is a vehicle control unit of an airbag system. The function of the first control unit 102 serves to receive an accident-related data in relation to the vehicle, in the event of a traumatic event, such as a vehicle collision. The second electronic control unit 104 may be a vehicle control unit for activating autonomous driving function. In certain embodiments, further electronic control unit may be considered for assisting autonomous driving function, such as vehicle control unit for navigation system may be included. This arrangement allows the vehicle to trigger autonomous driving function in the event of an accident. Examples of autonomous driving function may be self-driving or self-parking, to ensure the safety of the occupant(s) in the vehicle after a traumatic event happens. By way of example, the second control unit 104 may be triggered such that the vehicle may self-drive for a short distance, decelerate and come to a complete stop (self-park). Other functions may include turning on the hazard lights of the vehicle to inform other road users of the intention to decelerate. The telematic control unit 106 functions as a communication node between the vehicle and any external party, thereby allowing sending wireless communication signals from vehicle-to-vehicle or vehicle-to-anything, through suitable wireless communication protocol.

The gateway 108 is in wireless communication with at least one type of electronic sensing device adapted for collecting health data in relation to a user, through a wireless communication protocol 118. Suitable types of wireless communication protocol 118 may include wireless network (WiFi), Bluetooth technology, Bluetooth Low Energy (BLE) technology, Near Field Communication (NFC), ANT+, ZigBee, or even Body Area Network (BAN).

Examples of suitable sensing device includes mobile device 110 enabled with sensors for monitoring health data of user, wearable devices 112, which may be synchronized or paired with mobile device 110 for receiving relevant health data. The mobile device 110 may contain a computer-readable storage medium suitable for storing history of health data collected. In certain embodiment, the mobile device 110 is in wireless communication with an external server, suitable for receiving data or files transmitted from the mobile device 110 for storage purposes. The mobile device 110 may retrieve the stored data or files from the external server when necessary, by way of a wireless communication network 118.

Conventional sensors available in mobile device 110 and wearables 112 for health monitoring includes pulse, activity or exercise, sleep pattern, heart rate, oxygen level in blood and measurement of stress level. In one embodiment, health data from a mobile device 110 and a wearable device 112 provides sufficient information to generate a set of data comprising at least a part of the health data.

In another embodiment, the use of sensing device 114 such as vehicle interior sensors for receiving health data in relation to occupant(s) is preferred. Health data received by the gateway 108 is processed and a set of data comprising at least a part of the health data is generated in response to a traffic accident. For clarity and brevity, some examples of how vehicle interior sensors may assist to collect health data from an occupant within a cabin compartment of a vehicle is as described below, to support exemplary embodiments set forth in this disclosure.

Vehicle interior sensors 114 may be embedded within an interior of a cabin compartment of a vehicle to receive health data of occupants. Suitable location includes a driver's seat, a passenger seat, a seat belt, a steering wheel or a gear-changing joystick. For example sensor or sensing devices may be embedded in a driver's seat or passenger's seat to measure activity of the occupant. Another example may be measuring a temperature of the driver or passenger by using a thermal camera which can be embedded within a human-machine interface such as a display or an instrument cluster. An alternative example is to embed a sensor in the seat belt for measurement of heart rate or amount of perspiration of the occupant for instance. In certain embodiments, the sensors may be embedded in headrest or head gear, for collecting health data in relation to electroencephalogram (EEG). It shall be understood, the above examples are non-exhaustive and merely supports exemplary embodiments of this disclosure.

The health data received from the vehicle interior sensors 114 is in wireless communication with the gateway 108 using wireless communication protocol 118, examples of which are as listed above. Suitable vehicle interior sensors 114 may be bio sensors, for example, amperometric biosensor, blood glucose biosensor, potentiometric biosensor, conduct metric biosensor, thermometric biosensor, optical biosensor, fibre optic lactate biosensor, piezoelectric biosensors, immune-biosensors or any combination thereof.

In another embodiment involving multiple occupants, for example a public transport 414, an occupant may be required to register his presence in the vehicle 414 by tapping a RFID card at a terminal electrically connected to the system 100. Upon registration, a pairing process is necessary to pair and connect the mobile device 110, 112 of the occupant with the gateway 108 in order for the system 100 to verify an identification of the occupant on board the vehicle 414, and allow wireless communication of health data between the mobile device 110, 112 and the gateway 108.

In one embodiment, the occupant may be allocated a seat. In this embodiment, the occupant is required to scan a machine readable code at a terminal electronically connected to the system 100 of the vehicle 414. An example of a machine readable code may be a QR code which is printed on a public transport ticket, containing an identification code of the occupant. Upon scanning the identification code of the occupant at the terminal, the gateway 108 and the allocated seat of the occupant is considered paired. Successfully pairing and connecting to the mobile device 110, 112 of the occupant to the gateway 108 allows collection of health data, by the gateway, by way of the vehicle interior sensors 114 embedded within the allocated passenger seat. In the event of a vehicle collision, the first control unit 102 receive an accident-related data in relation to the vehicle, and the at least one gateway 108 initiates collecting the health data of each occupant. The health data collected with respect to each occupant is classified according to the identification of the occupant.

In another embodiment, the occupant does not has an allocated seat. In order to pair and connect the mobile device 110, 112 of the occupant with the gateway 108, the occupant is required to tap his RFID card with his wearable device 112. The RFID card contains an unique identifier of the occupant. An example of an unique identifier may be a fingerprint of the occupant. This pairing process may be enabled by short-range communication mode, such as near-field communication (NFC) or Bluetooth Low Energy (BLE) technology. Successfully pairing and connecting the mobile device 110, 112 to the gateway 108 enables the mobile device 110, 112 of the occupant to wirelessly communicate health data collected by the mobile device 110, 112 with gateway 108. In the event of a vehicle collision, the unique identifier of the occupant facilitates the gateway 108 to classify the health data collected according to the identification of the occupant(s), to generate a set of health data to be communicated to the predetermined external in the event of a traffic accident.

In yet another exemplary embodiment, an occupant without an allocated seat may pair and connect the mobile device 110,112 to the gateway 108 by way of entering details of his identification. The entering of identification may be by way of a mobile application specifically designed to work with the system 100. By successfully pairing the mobile device 110, 112 and connecting the gateway 108, the health data collected by the mobile device 110, 112 may be wirelessly communicated to the gateway 108. The gateway 108 may thereby classify the health data collected according to the identification of the occupant(s) to generate a set of health data to be communicated to the predetermined external agent in the event of a traffic accident.

In one embodiment, upon successfully pairing and connecting the mobile device 100 to the gateway 108 enables an access right to an external server 416. The external server 416 may be a computer-readable storage medium for storing a history of health data. The access right from the mobile device 110, allows the gateway 108 to request for a history of health data records with respect to an identified occupant after a traffic accident. The history of health data records may be a part of the set of health data to be communicated to the predetermined external agent in the event of a traffic accident.

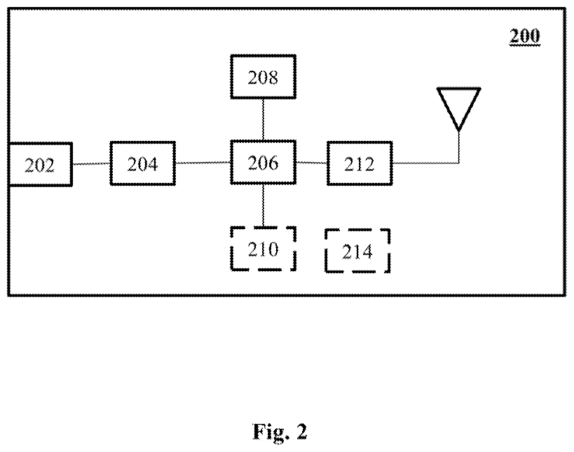

An exemplary gateway contain hardware elements as shown in block diagram 200 in FIG. 2. A connector interface 202 allows the gateway 108 to be connected to the wired communication protocol 116 of a vehicle network. The gateway 108 includes a transceiver 204, for transmitting and receiving signals routed via the wired network protocol 116. A microprocessor or a micro-controller unit 206 function to process the health data received from the mobile device 110, such as a mobile communication device, wearable device 112, and/or vehicle interior sensors 114. In the present disclosure, the microprocessor 206 may be configured to enable one or more methods, processes and/or operations of system 100. Examples of such methods, processes and operations include operable to receive and process health data, classify health data, automate reasoning and execute algorithmic functions, but the present disclosure is not limited thereto.

The gateway 108 includes a computer-readable storage medium or database for storing health data collected from the mobile device 110, wearable device 112, vehicle interior sensors 116 and RFID card. Additionally, a wireless transceiver 212 adapted for transmitting and receiving wireless signals shall be included, to enable the gateway 108 to receive wireless signals from the mobile device 110, wearable 112 and vehicle interior sensors 114. A wireless signal may be classified as long-range or short-range. Examples of short-range wireless communication signals includes radio frequency (RF) signals, ultra-wide band (UWB) signals, infrared signals or typically signals that conforms with the standards of near field communication (NFC) protocol, Bluetooth Low Energy (BLE) protocol, vehicle to everything (V2X) protocol, dedicated short-range communications (DSRC) protocol, direct-sequence spread spectrum (DSSS) protocol, wireless fidelity (WiFi) protocol or wireless local area network (WLAN) protocol. Types of long-range wireless signals include Bluetooth protocol, ultra-wide band (UWB), general packet radio service (GPRS), universal mobile telephone system (UMTS), 3G, 4G, 5G or any other types of suitable long-range wireless connectivity or connections. A suitable wireless transceiver may be a transceiver adapted for receiving Bluetooth or Bluetooth Low Energy wireless signals. Optionally, the gateway 108 may include a security element 210 to prevent malicious attack on the gateway 108. An example of a security element 210 may be a set of policy or a set of instructions that is pre-programmed in the micro-controller unit 206. In the alternative, it can also be a hardware module that function to perform encryption and decryption. In certain embodiments, the security element 210 may be a combination of both hardware and software. An auxiliary wireless transceiver 214 may also be included, to receive alternative forms of wireless signals apart from Bluetooth or Bluetooth Low Energy, for example near-field communication.

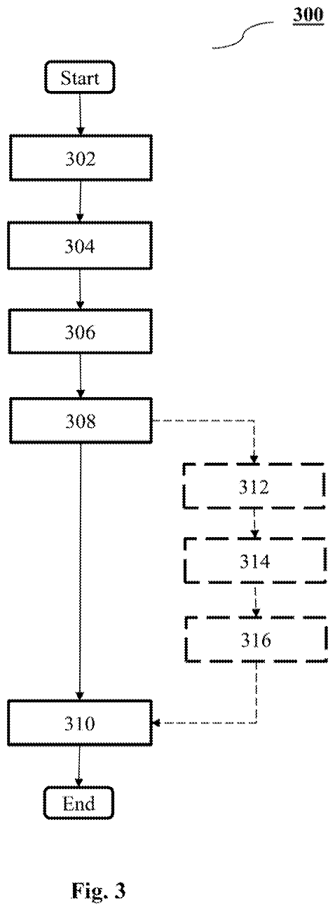

FIG. 3 illustrates a flowchart 300 explaining the process for processing health information after a traumatic or crisis event, such as an occurrence of traffic accident or vehicle collision. In the step 302, the first electronic control unit 102 receives an accident-related data in relation to the vehicle. In response to the accident-related data received, the first electronic control unit 102 communicate, route or transmit the accident-related data to a gateway 108 in the step 304, via the wired communication protocol.

In the next step 306, the gateway 108 initiates collecting at least one health data of an occupant in the vehicle from the mobile device 110, the wearable device 112, and the vehicle interior sensors 114. The gateway 108 begins to process the health data collected and prepares a set of data to be transmitted to the telematics control unit 106. A first signal to the telematics control unit 106 at the step 308, where the first signal carries the information of the set of data, containing at least one type of health data of the occupant(s) in the vehicle. The types of health data includes pulse rate, body temperature, respiration rate, blood pressure, blood glucose level, electroencephalogram (EEG) and/or electromyography (EMG) of the occupant(s).

In response to receiving the first signal by the telematics control unit 106, at the step 310, the telematics control unit 106 transmit an alert and the set of data wirelessly to a predetermined external agent in the step 310. The predetermined external agent may be an emergency response team, an emergency medical care giver and/or a medical communal centre, for example a hospital. This step allows the processing of health information collected to be communicated to the predetermined external party, who may be better prepared to provide adequate medical attention to the vehicle crash victim(s) in a timely manner.

In another embodiment, optional step 312 includes transmitting a second signal from the gateway 108 to the second electronic control unit 104, to trigger autonomous driving function, for instance, self-driving and/or self-parking, to ensure safety of the occupant(s) depending on road condition, as well as turning on of hazard light indicator to inform other road users the vehicle is decelerating and coming to a complete halt.

In yet another embodiment, optional step 314 includes transmitting a positional data of the vehicle in the set of data to be communicated to the predetermined external party. Provisioning the positional data of the vehicle in the set of data transmitted to the predetermined external party allows the emergency response team to deploy resources to the exact location of vehicle collision scene. In certain embodiments, an on-board navigation system is necessary for determining the positional data of the vehicle. In other embodiments, the use of global positioning system (GPS) of the mobile device 110 may trigger the positional data of the vehicle.

In a further embodiment, optional step 316 includes transmitting an identification of an occupant in the set of data to be communicated to the predetermined external party. The identification of the occupant may be communicated together with the health data of classified in accordance to the identification of the occupant, to the predetermined external party. It shall be understood by a practitioner skilled in the art, the omission of optional steps 312, step 314 and step 316 shall not depart from the scope and spirit of this disclosure.

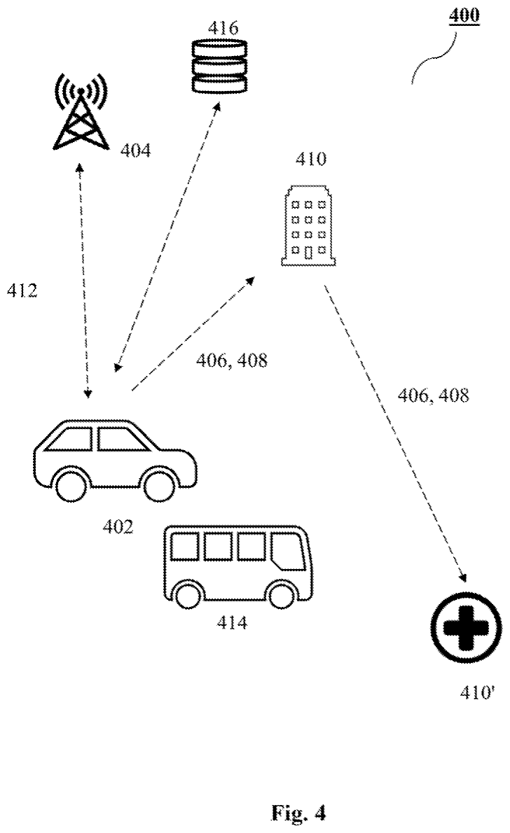

FIG. 4 illustrates an infrastructure 400 according to a preferred embodiment as disclosed herein. The arrows as shown in FIG. 4 are meant for illustrating the flow of information between different nodes of the infrastructure 400.

Vehicle 402, 414 is equipped with a system 100 (FIG. 1 referred) for processing health information as disclosed herein. In the event of a vehicle collision, the first electronic control unit 102 on-board vehicle 402 receives an accident-related data in relation to the vehicle 402, and in response to the accident-related data received, the first electronic control unit 102 transmit the accident-related data to gateway 108 on-board vehicle 402 via the vehicle's wired communication protocol 116. In response to the accident-related data received, the gateway 108 initiates collecting at least one health data of an occupant on-board the vehicle 402 from the occupant's mobile device, 110, 112 and/or sensing device equip in the vehicle 402, 414 via wireless communication protocol 118.

The health data received by the gateway 108 may be further processed by a microprocessor or micro-controller unit 206, before gateway 108 transmit a first signal to the telematic control unit 106. In response to receiving the first signal, the telematic control unit 106 wirelessly transmits an alert 406 and a set of data 408 to a predetermined external agent 410, via a satellite 404. The predetermined external agent 410 may be an emergency response team 410′, an emergency medical care giver and/or a medical communal centre, for example a hospital. In certain embodiments, the set of data includes a positional data 412 of the vehicle 402, such that the emergency response team is able to reach the location of accident and provide immediate medical attention to the vehicle collision victim. In certain embodiment, the positional data 412 may be determined by using an on-board vehicle navigation system or global positioning system.

In some embodiments as discussed above, there may be more than one occupant on-board the vehicle, for example a public transport 414. In order for the gateway 108 to identify health data collected in relation to the respective occupants, registration of the occupant in the public transport 414 is required in order for the system 100 to recognise and classify the health data collected by the gateway 108 with respect to each of the occupant in the public transport 414.

For brevity, the registration process may involve carrying out pairing the mobile device 110, 112 of the occupant with the gateway 108 equipped in the vehicle 402, 414, of which details of the pairing process between mobile device 110, 112 of the occupant with the gateway 108 as discussed above shall be referred to.

In one embodiment, the gateway 108 is granted an access to an external server 416 which stores a history of health data of the occupants. The gateway 108 may initiate a request for the history of health data and transmit the history of heath data as a part of the set of health data to be communicated to the predetermined external agent 410, 410′.

The detailed description of the disclosure will be provided for the purpose of explaining the principles of the disclosure and its practical application, thereby enabling other skilled practitioner to understand the disclosure. Modifications and equivalents will be apparent to practitioners skilled in this art and are encompassed within the spirit and scope of the appended claims.

### LIST OF REFERENCE SKINS

100—system block diagram
102—first electronic control unit
104—second electronic control unit
106—telematics control unit
108—gateway
110—mobile communication device
112—wearable device
114—vehicle interior sensors
116—wired communication protocol
118—wireless communication protocol
200—gateway block diagram
202—connector interface
204—transceiver
206—microprocessor or micro-controller unit
208—computer-readable storage medium
210—security element
212—wireless transceiver
214—auxiliary wireless transceiver
300—flowchart
302—step of receiving an accident-related data
304—step of transmitting the accident-related data
306—step of collecting at least one health data
308—step of transmitting a first signal to telematic control unit;
310—step of transmitting an alert and a set of data
312—step of transmitting a second signal to second control unit;
314—step of transmitting a positional data
400—Infrastructure
402, 414—vehicle
404—satellite
406—an alert
408—a set of data
410—predetermined external agent
412—positional data
416—external server

The invention claimed is:

1. An information processing system for vehicle comprising:
a first electronic control unit equipped in a vehicle, the first electronic control unit configured to receive and transmit an accident-related data in relation to the vehicle; and
at least one gateway configured to receive the accident-related data from the first electronic control unit, to receive at least one health data of an occupant in the vehicle, the at least one health data stored in or collected by at least one of mobile device of the occupant and sensing device embedded in the vehicle; and transmit a first signal to a telematic control unit upon the receipt of the accident-related data; and
the telematic control unit configured to receive the first signal from the at least one gateway and transmit an alert together with a set of data which comprises at least a part of the health data or an access right to an external server comprising at least a part of the health data to a predetermined external agent upon the receipt of the first signal.

2. The system according to claim 1, wherein the at least one gateway is further configured to transmit the at least a part of the health data to the telematic control unit.

3. The system according to claim 1, wherein the gateway is further configured to transmit a second signal to a second electronic control unit upon the receipt of the accident-related data, and further comprising the second electronic control unit configured to receive the second signal from the gateway and activate one or more autonomous driving functions for emergency situation upon the receipt of the second signal.

4. The system according to claim 3, wherein the autonomous driving function is selected from a self-driving function, a self-parking function, a hazard light indicator function or any combination thereof.

5. The system according to claim 1, wherein the at least one health data is transmitted via wired communication protocol or wireless communication protocol by the at least one of mobile device of the occupant and sensing device embedded in the vehicle to the gateway.

6. The system according to claim 1, wherein the accident-related data comprises a vehicle collision information.

7. The system according to claim 1, wherein the mobile device of the occupant is selected from a mobile communication device, a wearable device, or any combination thereof.

8. The system according to claim 7, wherein the mobile device of the occupant is configured for pairing with the gateway, prior to receiving at least one health data of the occupant in the vehicle.

9. The system according to claim 7, wherein the pairing between the mobile device of the occupant and the gateway is conducted by scanning an identification code of the occupant at a terminal electrically connected to the system of the vehicle.

10. The system according to claim 7, wherein the pairing between the mobile device of the occupant and the gateway is conducted by tapping a radio frequency identification card containing an unique identifier of the occupant.

11. The system according to claim 7, wherein the pairing between the mobile device of the occupant and the gateway is conducted by entering an identification of the occupant by way of the mobile device.

12. The system according to claim 8, wherein the pairing between the mobile device of the occupant and the gateway is configured to enable wireless communication health data between the mobile device of the occupant and the gateway upon successfully pairing.

13. The system according to claim 1, wherein the gateway is configured to wirelessly communicate with an external server using a long-range communication mode.

14. The system according to claim 13, wherein the external server is a computer-readable storage medium configured to store the health data from the gateway.

15. The system according to claim 1, wherein the sensing device comprises a vehicle interior sensor selected from the group consisting of, an amperometric biosensor, a blood glucose biosensor, a potentiometric biosensor, a conduct metric biosensor, a thermometric biosensor, optical biosensor, a fibre optic lactate biosensor, a piezoelectric biosensors, an immune-biosensors or any combination thereof.

16. The system according to claim 1, wherein the at least one health data comprises pulse rate, body temperature, respiration rate, blood pressure, blood glucose level, electroencephalogram, electromyography or any combination thereof.

17. The system according to claim 1, wherein the set of data further comprises a positional data of the vehicle.

18. The system according to claim 1, wherein the set of data further comprises the identification of at least one occupant in the vehicle.

19. A method of processing information for vehicle, the method comprising:
receiving, by a first electronic control unit of the vehicle, an accident-related data in relation to the vehicle;
transmitting, by the first electronic control unit, the accident-related data to at least one gateway of the vehicle;
receiving, by the at least one gateway, at least one health data of an occupant in the vehicle from at least one mobile device of the occupant and sensing device embedded in the vehicle;
transmitting, by the at least one gateway, a first signal to a telematics control unit of the vehicle; and
transmitting, by the telematics control unit, upon the receipt of the first signal, an alert and a set of data comprising at least a part of the health data collected by the at least one gateway, or an access right to an external server, to a predetermined external agent.

20. The method according to claim 19, wherein the at least a part of the health data is transmitted to the telematics control unit by the at least one gateway.

21. The method according to claim 20, further comprising transmitting, by the at least one gateway, a second signal to a second electronic control unit of the vehicle upon the receipt of the accident-related data, for activating one or more autonomous driving functions for emergency situation upon receiving the second signal.

22. The method according to claim 19, further comprising collecting, by the at least one gateway, a positional data of the vehicle upon the receipt of the accident-related data, wherein the set of data further comprises the positional data of the vehicle.

23. The method according to claim 19, further comprising collecting, by the at least one gateway, an identification of at least one occupant in the vehicle, upon the receipt of the accident-related data, wherein the set of data further comprises the identification of at least one occupant in the vehicle.

24. The method according to claim 19, wherein the at least one health data is transmitted via wired communication protocol or wireless communication protocol by the at least one of mobile device of the occupant and sensing device to the gateway.

25. A non-transitory computer-readable storage medium storing instructions that, where executed by one or more processors, cause the processors to perform operations of the method according to claim 19.
